(19) United States
(12) Patent Application Publication
(10) Pub. No.: US 2024/0027434 A1
(43) Pub. Date: Jan. 25, 2024

(54) METHOD FOR QUANTIFYING CPG-CONTAINING OLIGONUCLEOTIDES IN COMPOSITIONS COMPRISING ALUM

(71) Applicant: Dynavax Technologies Corporation, Emeryville, CA (US)

(72) Inventor: Martin GOHLKE, Berkeley, CA (US)

(73) Assignee: Dynavax Technologies Corporation, Emeryville, CA (US)

(21) Appl. No.: 18/267,083

(22) PCT Filed: Dec. 14, 2021
(86) PCT No.: PCT/US2021/072922

§ 371 Date: Jun. 13, 2023

Related U.S. Application Data
(60) Provisional application No. 63/126,403, filed on Dec. 16, 2020.

(51) Int. Cl. G01N 33/53; C12Q 1/6806; G01N 33/68

(52) U.S. Cl. CPC G01N 33/5308; C12Q 1/6806; G01N 33/6842; G01N 2440/14

(57) ABSTRACT

The present disclosure relates to methods for characterizing formulations comprising aluminum hydroxide particles (alum), an antigen bound to the alum, and an unmethylated cytidine-phospho-guanosine-containing oligodeoxynucleotide (CpG ODN). In particular, the present disclosure provides methods for determining concentration of CpG ODN in a vaccine formulation through use of a colorimetric assay for measuring total phosphorus.

### CROSS-REFERENCE TO RELATED APPLICATIONS

This application claims priority to and the benefit of U.S. Provisional Application No. 63/126,403, filed Dec. 16, 2020, the disclosure of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 377882007540SEQLIST.TXT, date recorded: Dec. 13, 2021, size: 984 bytes).

### FIELD

The present disclosure relates to methods for characterizing formulations comprising aluminum hydroxide particles (alum), an antigen bound to the alum, and an unmethylated cytidine-phospho-guanosine-containing oligodeoxynucleotide (CpG ODN). In particular, the present disclosure provides methods for determining concentration of CpG ODN in a vaccine formulation through use of a colorimetric assay for measuring total phosphorus.

### BACKGROUND

Adjuvants are substances that are combined with antigen(s) in a vaccine to enhance immune responses against the antigen(s). Adjuvants are typically used in vaccines where an antigen from a microbe is insufficient on its own to generate a protective immune response against the microbe. Oligodeoxynucleotides, such as CpG 1018, containing unmethylated cytidine-phospho-guanosine motifs (CpG ODN) stimulate toll-like receptor 9 (TLR9)-dependent immune responses. CpG 1018 is the adjuvant found in HEPLISAV-B (hepatitis B vaccine marketed by Dynavax Technologies Corp., Emeryville, CA), which has been shown to generate faster, stronger and longer-lasting protective vaccine responses than a hepatitis B vaccine formulated with an aluminum salt adjuvant (Schillie et al., MMWR, 67:455-458, 2018).

In some instances, it may be desirable to include both an aluminum salt adjuvant and a CpG ODN adjuvant, as well as an antigen in a vaccine formulation. It is important to carefully characterize vaccine formulations, particularly those intended for administration to human subjects. Thus, there is a need in the art for methods for determining concentration of CpG ODN in a vaccine formulation.

### SUMMARY

The present disclosure relates to methods for characterizing formulations comprising aluminum hydroxide particles (alum), an antigen bound to the alum, and an unmethylated cytidine-phospho-guanosine-containing oligodeoxynucleotide (CpG ODN). In particular, the present disclosure provides methods for determining concentration of CpG ODN in a vaccine formulation through use of a colorimetric assay for measuring total phosphorus.

### DETAILED DESCRIPTION

The present disclosure relates to methods for characterizing formulations comprising aluminum hydroxide particles (alum), an antigen bound to the alum, and an unmethylated cytidine-phospho-guanosine-containing oligodeoxynucleotide (CpG ODN). In particular, the present disclosure provides methods for determining concentration of CpG ODN in a vaccine formulation through use of a colorimetric assay for measuring total phosphorus.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 jig of CpG 1018 refers to 2700 jig to 3300 jig of CpG 1018).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG”, “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo. Examples of measurable immune responses include, but are not limited to, antigen-specific antibody production, secretion of cytokines, activation or expansion of lymphocyte populations, such as NK cells, CD4+T lymphocytes, CD8+T lymphocytes, B lymphocytes, and the like. Preferably, the CpG oligonucleotide preferentially activates a Th1-type response.

The term “aluminum salts” as used herein, refer to a class of aluminum-containing inorganic chemical compounds suitable for use as a vaccine adjuvant to increase the desired immune response to a vaccine antigen (e.g., generating antibodies and/or inducing cell-mediated immunity against a simultaneously administered antigen).

The terms “polypeptide” and “protein” are used interchangeably herein, and encompass both short chains (from 8 to 50) and long chains (above 50) of amino acids linked by peptide bonds. “Polypeptide antigens” can include purified native peptides, synthetic peptides, recombinant peptides, or fragments of such peptides. Polypeptide antigens are preferably at least 8 amino acid residues in length, preferably from 8 to about 1800 amino acids in length, more preferably from about 10 to about 1000 amino acids in length. In some embodiments, the polypeptide is at least (lower limit) 8, 9, 10, 11, 12, 13, 14, 15, 16, 17, 18, 19, 20, 25, 30, 35, 40, 50, 60, 70, 80 or 90 amino acids in length. In some embodiments, the polypeptide is at most (upper limit) 1800, 1700, 1600, 1500, 1400, 1300, 1200, 1100, 1000, 900, 800, 700, 600, 500, 400, 300, 250, 200, 150 or 100 amino acids in length.

An “effective amount” or a “sufficient amount” of a substance is that amount sufficient to effect beneficial or desired results, including clinical results, and, as such, an “effective amount” depends upon the context in which it is being applied. In the context of administering an formulation, an effective amount contains sufficient antigen and TLR9 agonist to stimulate an immune response (preferably a seroprotective level of antibody to the antigen).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an formulation refers to a measured portion of the formulation taken by (administered to or received by) a subject (e.g., human subject) at any one time.

The term “isolated” as used herein refers to a compound that is removed from at least one component with which it is naturally associated (e.g., separated from its original environment). The term “isolated,” when used in reference to a recombinant protein, refers to a protein that has been removed from the cell culture (e.g., culture medium and host cells) that produced the protein. “Isolated” compounds are at least 75% free, preferably at least 90% free, more preferably at least 95% free (e.g., at least 95%, 96%, 97%, 98%, or 99% free) from other components with which they are associated. The term “isolated,” when used in reference to protein antigens, encompasses both purified native proteins, as well as recombinant or synthetic proteins.

### I. Formulations

Formulations of the present disclosure are for stimulating an immune response against an antigen. The formulations comprise an antigen, an aluminum salt adjuvant to which the antigen is adsorbed, and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the aluminum salt adjuvant comprise aluminum hydroxide particles (alum). The antigen and the oligonucleotide are present in the formulation in amounts effective to stimulate an immune response against the antigen in a mammalian subject, such as a human subject.

A. Toll-Like Receptor 9 (TLR9) Agonists

Toll-like receptors (TLRs) are expressed in and on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, CA) have identified a 22-mer phosphorothioate linked oligodeoxynucleotide, CpG 1018, which contains specific sequences that can substantially enhance the immune response to co-administered antigens across species (Campbell, Methods Mol Biol, 1494:15-27, 2017). CpG 1018 (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018 is a CpG-B ODN that is active in mice, rabbits, dogs, baboons, cynomolgus monkeys, and humans Thus in some preferred embodiments, the TLR9 agonist is an oligonucleotide comprising the sequence of SEQ ID NO:1.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical formulation comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the formulation, which is typically a 0.5 ml dose, may comprise from about 375 jig to about 6000 jig of the CpG oligonucleotide, preferably from about 500 jig to about 5000 jig of the CpG oligonucleotide, preferably from about 750 jig to about 3000 jig of the CpG oligonucleotide. In some embodiments, a 0.5 ml dose of the formulation comprises greater than about 250, 500, 750, 1000, or 1250 jig of the CpG oligonucleotide, and less than about 6000, 5000, 4000, 3000, or 2000 jig of the CpG oligonucleotide. In some embodiments, a 0.5 ml dose of the formulation comprises about 750, 1500, 3000, or 6000 jig of the CpG oligonucleotide. In some embodiments, a 0.5 ml dose of the formulation comprises about 750 jig of the CpG oligonucleotide. In some embodiments, a 0.5 ml dose of the formulation comprises about 1500 jig of the CpG oligonucleotide. In some embodiments, a 0.5 ml dose of the formulation comprises about 3000 jig of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

B. Aluminum Salt Adjuvants

When used in vaccines, aluminum salts are typically wet-gel suspensions of irregularly-shaped and sized particles that possess crystalline structures of any one of several polymorphs. The aluminum salt adjuvant of the formulations of the present disclosure is an aluminum hydroxide adjuvant. An exemplary aluminum hydroxide adjuvant is ALHYDROGEL, a vaccine adjuvant marketed by Croda International PLC (East Yorkshire, UK). However, the present disclosure is in no way limited to the use of this brand of aluminum hydroxide adjuvant. Other brands and non-branded aluminum-containing adjuvants are suitable for use in the formulations described herein, provided they have comparable physiochemical properties. Such properties, which are compatible with incorporation into human vaccines, include: 500-10,000 nm diameter size range; porous crystalline structure; and net positive surface charge. Aluminum hydroxide particles (alum) have a net positive chart at pH 5-7 and therefore are capable of adsorption of a negatively charged antigen (Huang and Wang, Int J Pharm, 466(1-2):139-146, 2014) and a negatively charged CpG ODN. In some embodiments, a unit dose (e.g., about 0.5 ml) of the formulation comprises from about 0.25 to about 0.50 mg Al³⁺, preferably from about to about 0.40 mg Al³⁺.

C. Antigens

The antigen of the formulations of the present disclosure comprise an isolated polypeptide, polysaccharide, glycoprotein, and/or a conjugate of a polysaccharide bound to a carrier protein. As such, the formulations of the present disclosure are subunit vaccines that are devoid of whole cells. Said another way, the formulations of the present disclosure are not inactivated whole cell vaccines and are not recombinant DNA or RNA vaccines. The DNA and/or RNA of whole cells (e.g., microbes or cancer cells) or vectors is expected to interfere with the use of total phosphate as a proxy for determination of the CpG ODN content of the formulations in the methods of the present disclosure. A unit dose of the formulation, which is typically a 0.5 ml dose, may comprise about 1 μg to about 1 mg of the isolated antigen, preferably about 5 μg to about 500 jig of the antigen, preferably about 10 jig to about 100 jig of the antigen, or preferably about 15 jig to about 75 jig of the antigen.

D. Additional Components

The formulations of the present disclosure may comprise one or more additional components, such as one or more excipients, another adjuvant, and/or additional antigens.

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the formulations may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the formulations comprise an aqueous vehicle as a solvent. Suitable vehicles include for instance sterile water, saline solution, phosphate buffered saline, and Ringer's solution. In some embodiments, the formulation is isotonic.

The formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the formulation within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The formulations may comprise a tonicity adjusting agent. Suitable tonicity adjusting agents include for instance dextrose, glycerol, sodium chloride, glycerin and mannitol.

The formulations may comprise a bulking agent. Bulking agents are particularly useful when the pharmaceutical formulation is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The formulations may comprise a preservative. Suitable preservatives include for instance antioxidants and antimicrobial agents. However, in preferred embodiments, the formulation is prepared under sterile conditions and is in a single use container, and thus does not necessitate inclusion of a preservative.

### II. Methods

The present disclosure relates to methods for characterizing formulations comprising a CpG ODN by use of a colorimetric assay for measuring total phosphate. Colorimetic assays for measuring total phosphate have been described (Ames, Methods in Enzymology, 8:115-118, 1966; and U.S. Environmental Protection Agency, Method 365.3, 1978), but scientific publications describing the use of such assays for measuring CpG ODN in a vaccine formulation have not been found. Thus, the present disclosure provides novel methods for characterizing vaccine formulations, comprising:
a) mixing sulfuric acid and ammonium persulfate with a formulation comprising aluminum hydroxide particles (alum), an antigen bound to the alum, and a CpG-containing oligodeoxynucleotide (CpG ODN) to convert organic phosphorus in the CpG ODN to orthophosphate in a mixture;
b) reacting the orthophosphate with ammonium molybdate-antimony potassium tartrate to form an antimony-phosphate-molybdate complex;
c) contacting the antimony-phosphate-molybdate complex with ascorbic acid to form a blue-colored complex in which the blue color is proportional to concentration of total phosphorus in the complex;
d) measuring absorbance at 650 nm with a spectrophotometer;
e) calculating the concentration of total phosphorus in the complex from a standard curve generated by plotting absorbance values of standards versus phosphorus concentrations of the standards; and
f) determining concentration of CpG ODN in the formulation from the concentration of total phosphorus. Typically, the CpG ODN of the formulation comprises bound CpG ODN (bound to alum), and unbound CpG ODN (not bound to alum). In some embodiments, the methods further comprise filtering the orthophosphate-containing mixture of step a) to produce a filtrate comprising the orthophosphate, prior to reacting the orthophosphate in step b). In some embodiments, the methods further comprise centrifuging the orthophosphate-containing mixture of step a) to produce a supernatant comprising orthophosphate prior to reacting the orthophosphate in step b). Absent a fractionation step prior to step b), the concentration of CpG ODN determined by the methods of the present disclosure represents the concentrations of both bound CpG ODN and unbound CpG ODN.

In instances in which it is desirable to determine concentration of CpG bound to alum in the formulation and/or concentration of unbound CpG in the formulation, or a ratio of CpG bound to alum to unbound CpG in the formulation, the methods further comprise a step of centrifuging the formulation to produce a supernatant comprising unbound CpG, and a pellet comprising the antigen and the CpG bound to the alum, and separately subjecting the supernatant and/or the pellet to steps a)-d) of the methods of the preceding paragraph.

### ENUMERATED EMBODIMENTS

1. A method comprising:
a) providing a formulation comprising aluminum hydroxide particles (alum), an antigen bound to the alum, and a CpG-containing oligodeoxynucleotide (CpG ODN);
a) mixing sulfuric acid and ammonium persulfate with a formulation comprising aluminum hydroxide particles (alum), an antigen bound to the alum, and a CpG-containing oligodeoxynucleotide (CpG ODN) to convert organic phosphorus in the CpG ODN to orthophosphate in a mixture;
b) reacting the orthophosphate with ammonium molybdate-antimony potassium tartrate to form an antimony-phosphate-molybdate complex;
c) contacting the antimony-phosphate-molybdate complex with ascorbic acid to form a blue-colored complex in which the blue color is proportional to concentration of total phosphorus in the complex;
d) measuring absorbance at 650 nm with a spectrophotometer;
e) calculating the concentration of total phosphorus in the complex from a standard curve generated by plotting absorbance values of standards versus phosphorus concentrations of the standards; and
f) determining concentration of CpG ODN in the formulation from the concentration of total phosphorus.

2. The method of embodiment 1, wherein step a) comprises boiling, cooling and filtering the mixture.

3. The method of embodiment 1 or embodiment 2, wherein steps a)-c) are conducted in acid-washed glassware.

4. The method of any one of embodiments 1-3, wherein step d) is conducted within to 60 minutes of completion of step c).

5. The method of any one of embodiments 1-4, wherein the formulation comprises saline.

6. The method of any one of embodiments 1-4, wherein the formulation comprises a buffer having a pH in a range of about 6 to about 9, optionally having a pH in a range of 6.5 to 8.5, or 7 to 8.

7. The method of embodiment 6, wherein the buffer is devoid of phosphorus, optionally wherein the buffer is selected from the group consisting of an acetate buffer, a bicarbonate buffer, a borate buffer, a carbonate buffer, a citrate buffer, a glycine buffer, a phthalate buffer, a tetraborate buffer, and a TRIS buffer.

8. The method of embodiment 6, wherein the buffer is a phosphate buffer and the method further comprises subtracting phosphate concentration of the buffer from the total phosphate concentration before step f).

9. The method of any one of embodiments 1-8, wherein the CpG ODN is from 8 to nucleotides in length comprising an unmethylated cytidine-phospho-guanosine (CpG) motif, optionally wherein the CpG ODN is from 10 to 35 nucleotides in length.

10. The method of embodiment 9, wherein the CpG ODN comprises the sequence (SEQ ID NO:3).

11. The method of embodiment 9, wherein the CpG ODN comprises the sequence of

(SEQ ID NO: 1)
5′-TGACTGTGAA CGTTCGAGAT GA-3′.

12. The method of any one of embodiments 9-11, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.

13. The method of any one of embodiments 9-12, wherein the CpG ODN comprises at least one phosphorothioate linkage, optionally wherein all nucleotide linkages are phosphorothioate linkages.

14. The method of any one of embodiments 9-13, wherein the CpG ODN is double-stranded.

15. The method of any one of embodiments 1-14, wherein the antigen comprises at least one isolated polypeptide.

16. The method of embodiment 15, wherein the at least one polypeptide is from 8 to about 1800 amino acids in length.

17. The method of embodiment 15 or embodiment 16, wherein the antigen is a viral antigen, a bacterial antigen, or a fungal antigen.

18. The method of embodiment 15 or embodiment 16, wherein the antigen is a tumor antigen.

19. The method of any one of embodiments 15-18, wherein the antigen is a fusion protein comprising two or more polypeptides, wherein each polypeptide comprises amino acid sequences from different antigens or non-contiguous amino acid sequences from the same antigen.

20. The method of any one of embodiments 1-19, wherein the formulation comprises a heterogeneous mixture of particles in which the ratio of the CpG ODN to the alum is within the range of from about 1:10 to about 10:1 (weight/weight), and the ratio of the antigen to the alum is within the range of from about 1:10 to about 10:1 (weight/weight), optionally wherein the ratio of the CpG ODN to the alum and the ratio of the antigen to the alum are each within the range of from about 0.1 to about 1 (weight/weight).

21. The method of any one of embodiments 1-19, wherein the formulation of step b) is prepared by:
i) adding the antigen to the alum first, followed by adding the CpG ODN; or
ii) adding the antigen and the CpG ODN to the alum at the same time.

1. A method comprising:
a) providing a formulation comprising aluminum hydroxide particles (alum), an antigen bound to the alum, and a CpG-containing oligodeoxynucleotide (CpG ODN);
a) mixing sulfuric acid and ammonium persulfate with a formulation comprising aluminum hydroxide particles (alum), an antigen bound to the alum, and a CpG-containing oligodeoxynucleotide (CpG ODN) to convert organic phosphorus in the CpG ODN to orthophosphate in a mixture;
b) reacting the orthophosphate with ammonium molybdate-antimony potassium tartrate to form an antimony-phosphate-molybdate complex;
c) contacting the antimony-phosphate-molybdate complex with ascorbic acid to form a blue-colored complex in which the blue color is proportional to concentration of total phosphorus in the complex;
d) measuring absorbance at 650 nm with a spectrophotometer;
e) calculating the concentration of total phosphorus in the complex from a standard curve generated by plotting absorbance values of standards versus phosphorus concentrations of the standards; and
f) determining concentration of CpG ODN in the formulation from the concentration of total phosphorus.

2. The method of claim 1, wherein step a) comprises boiling, cooling and filtering the mixture.

3. The method of claim 2, wherein steps a)-c) are conducted in acid-washed glassware.

4. The method of claim 3, wherein step d) is conducted within 5 to 60 minutes of completion of step c).

5. The method of claim 4, wherein the formulation comprises saline.

6. The method of claim 4, wherein the formulation comprises a buffer having a pH in a range of about 6 to about 9.

7. The method of claim 6, wherein the buffer is devoid of phosphorus.

8. The method of claim 6, wherein the buffer is a phosphate buffer and the method further comprises subtracting phosphate concentration of the buffer from the total phosphate concentration before step f).

9. The method of claim 1, wherein the CpG ODN is from 8 to 50 nucleotides in length comprising an unmethylated cytidine-phospho-guanosine (CpG) motif.

10. The method of claim 9, wherein the CpG ODN comprises the sequence (SEQ ID NO:3).

11. The method of claim 9, wherein the CpG ODN comprises the sequence of 5′-TGACTGTGAA CGTTCGAGAT GA-3′(SEQ ID NO:1).

12. The method of claim 9, wherein the oligonucleotide comprises a modified nucleoside, wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.

13. The method of claim 9, wherein the CpG ODN is double-stranded and comprises at least one phosphorothioate linkage.

14. The method of claim 1, wherein the antigen comprises at least one isolated polypeptide.

15. The method of claim 14, wherein the antigen is a viral antigen, a bacterial antigen, or a fungal antigen.

16. The method of claim 14, wherein the antigen is a tumor antigen.

17. The method of claim 15, wherein the antigen is a fusion protein comprising two or more polypeptides, wherein each polypeptide comprises amino acid sequences from different antigens or non-contiguous amino acid sequences from the same antigen.

18. The method of claim 1, wherein the formulation of step b) is prepared by:
i) adding the antigen to the alum first, followed by adding the CpG ODN; or
ii) adding the antigen and the CpG ODN to the alum at the same time.

19. The method of claim 16, wherein the antigen is a fusion protein comprising two or more polypeptides, wherein each polypeptide comprises amino acid sequences from different antigens or non-contiguous amino acid sequences from the same antigen.


SEQUENCE LISTING (3 sequences)

SEQ ID NO: 1 - DNA, 22 bp, Artificial Sequence
  Synthetic Construct
tgactgtgaacgttcgagatga

SEQ ID NO: 2 - DNA, 11 bp, Artificial Sequence
  Synthetic Construct
  misc_feature 3, 7, 11: 2′-deoxy-7-deazaguanosine
tcgaacgttcg

SEQ ID NO: 3 - DNA, 10 bp, Artificial Sequence
  Synthetic Construct
aacgttcgag